(19) United States
(12) Patent Application Publication
(10) Pub. No.: US 2026/0253730 A1
(43) Pub. Date: Aug. 27, 2026

(54) PARKINSON'S DISEASE SAMPLE FEATURE TYPING METHOD, FEATURE TYPING SYSTEM, AND SAMPLE CLASSIFICATION MODEL TRAINING METHOD BASED ON LOCAL FIELD POTENTIAL

(71) Applicant: Guangdong University of Technology, Guangzhou (CN)

(72) Inventors: Guofa Cai, Guangzhou (CN); Jiaxin Bao, Guangzhou (CN); Guoen Cai, Guangzhou (CN); Qinyong Ye, Guangzhou (CN); Dijun Zhong, Guangzhou (CN); Yuqi Zeng, Guangzhou (CN)

(21) Appl. No.: 19/433,061

(22) Filed: Dec. 25, 2025

(30) Foreign Application Priority Data
Feb. 21, 2025 (CN) 202510192860.8

(51) Int. Cl. G16H 50/20; G16H 10/60

(52) U.S. Cl. CPC G16H 50/20; G16H 10/60

(57) ABSTRACT

Disclosed are a Parkinson's disease sample feature typing method, a feature typing system, and a sample classification model training method based on a local field potential. The typing method includes: calculating a power spectral density of a Beta-band signal of a local field potential of a sample to obtain an average power spectral density curve, and performing peak analysis on the average power spectral density curve to obtain a peak power spectral density; separately modeling a periodic element and an aperiodic element of an average power spectrum to extract a periodic power spectrum and an aperiodic component; calculating a phase-amplitude coupling feature of the Beta-band signal of the local field potential of the sample; and performing feature clustering analysis, and outputting sample typing results. In the disclosure, a multidimensional data model is constructed, and classification accuracy and reliability are improved.

### CROSS-REFERENCE TO RELATED APPLICATION

This application claims priority of Chinese Patent Application No. 202510192860.8, filed on Feb. 21, 2025, the entire contents of which are incorporated herein by reference.

### TECHNICAL FIELD

The disclosure belongs to the field of neurosciences, and more specifically, relates to a Parkinson's disease (PD) sample feature typing method, a feature typing system, and a sample classification model training method based on a local field potential (LFP).

### BACKGROUND

PD, a common neurodegenerative disease, mainly affects middle-aged and aged people. Clinically, PD is characterized by its typical motor symptoms including tremor, myotonia, bradykinesia and postural instability, accompanied by non-motor symptoms including cognitive dysfunction, depression, and sleep disturbances. In spite of the similar clinical manifestations, PD shows significant heterogeneity among different individuals, making early diagnosis, disease typing, and personalized treatment a difficult problem in medical research. The global trend of population aging drives a progressive elevation in the prevalence of PD year to year, which exerts a heavy burden on society and families. Consequently, the exploration of different subtypes of PD, coupled with the delivery of more efficacious interventions and treatments based on precise typing, has become a critical research direction in the field of neurosciences.

Currently, the diagnosis of PD is predominantly dependent on clinical assessment tools. However, the clinical manifestations of PD show marked individual variability, and patients can manifest entirely disparate symptom combinations across the various phases of the disease, making it extremely difficult to classify and predict solely on the basis of clinical symptoms. Research indicates that the Beta-band LFP is closely associated with motor symptoms, particularly during motor control and planning processes, where changes in Beta wave activity can reflect the motor functional status of patients. Although certain progress is made in electroencephalogram signal research in PD, most existing studies focus on analyzing the correlation between electroencephalogram features and single clinical indicator, lacking comprehensive modeling that integrates multiple clinical features and neural activity data.

The existing invention patent with publication number of CN 116458898 A provides a feature extraction method and system for PD-associated depression. The method includes: acquiring an electroencephalogram signal, processing the electroencephalogram signal to obtain an LFP, and preprocessing the LFP to obtain an LFP to be processed; applying continuous wavelet transform to the LFP to be processed, and retaining alpha, low-beta, high-beta, and beta bands; extracting burst features from all the described bands, applying continuous wavelet transform to process the LFP, obtaining burst properties of the alpha, low-beta, high-beta, and beta bands, and utilizing a PD-associated depression feature extraction system to achieve high-precision classification of depression severity on the basis of the extracted burst feature. However, in this approach, only wavelet transform is used for burst feature extraction, resulting in insufficient comprehensiveness in feature extraction.

### SUMMARY

The disclosure provides a PD sample feature typing method, a feature typing system, and a sample classification model training method based on an LFP, to overcome the shortcomings in the prior art including the failure to comprehensively model multiple clinical features and neural activity data, and insufficiently comprehensive feature extraction.

A primary objective of the disclosure is to solve the above-described technical problems, and the technical solutions of the disclosure are as follows.

In a first aspect of the disclosure, a PD sample feature typing method based on an LFP is provided, including the following steps:
calculating a power spectral density of a Beta-band signal of an LFP of a sample to obtain an average power spectral density curve;
performing peak analysis on the average power spectral density curve to obtain a peak power spectral density;
separately modeling a periodic element and an aperiodic element of the average power spectral density curve to extract a periodic power spectrum and an aperiodic component;
calculating a phase-amplitude coupling (PAC) feature of the Beta-band signal of the LFP of the sample; and
performing feature clustering analysis using the peak power spectral density, the periodic power spectrum, the aperiodic component, and the PAC feature, and outputting sample typing results.

In an embodiment, a method for calculating the power spectral density of the Beta-band signal of the LFP of the sample is a Welch method, specifically including the following steps:
framing an original LFP signal x[n], and calculating a windowed signal in combination with a Hanning window function w[n], an expression being as follows:

$${x}_{i} [ n ] = x [ n + i \cdot N ] \cdot w [ n ]$$

where n represents a sample index within a frame, n∈[1,N−1], xᵢ[n] represents a windowed signal of an iₜₕ frame, x[n] represents an original signal, w[n] represents a Hanning window, and N represents a frame length;
applying discrete Fourier transform (FFT) to each frame of the windowed signal, and calculating a frequency-domain signal Xᵢ[f], an expression being as follows:

$${X}_{i} [ f ] = FFT ( {x}_{i} [ n ] )$$

where f represents a frequency, FFT represents discrete Fourier transform, and xᵢ[n] represents a windowed signal of an iₜₕ frame;
taking a squared modulus of a frequency-domain signal for each frame to obtain a power spectral density Pᵢ[f] of the iₜₕ frame, an expression being as follows:

$${P}_{i} [ f ] = {\vert {X}_{i} [ f ] \vert}^{2} ;$$

and
averaging power spectra of all frames to obtain an average power spectral density curve P_{avg}[f], an expression being as follows:

$${P}_{avg} [ f ] = \frac{1}{N} {{\sum }_{i = 1}}^{N} {P}_{i} [ f ]$$

where N is the number of frames.

In an embodiment, separately modeling the periodic element and the aperiodic element of the average power spectral density curve to extract the periodic power spectrum and the aperiodic component specifically includes the following steps:
fitting a periodic oscillating component of the power spectrum using a Gaussian function to obtain a periodic power spectral density P_{periodic}(f), an expression being as follows:

$${P}_{periodic} ( f ) = A \cdot {e}^{- \frac{{( f - CF )}^{2}}{2 \cdot {( BW / ( 2 \cdot \sqrt{2 \cdot ln ( 2 )} ) )}^{2}}}$$

where f represents a frequency, A represents an amplitude of an oscillation, CF represents a central frequency of the oscillation, and BW represents a bandwidth of the oscillation;
fitting the aperiodic element of the power spectrum using a 1/f-like function to obtain an aperiodic power spectral density P_{aperiodic}(f), an expression being as follows:

$${P}_{aperiodic} ( f ) = \frac{offset}{{f}^{exponent}}$$

where f represents a frequency, offset represents an offset of the aperiodic element, and exponent represents an exponent of the aperiodic element;
constructing a complete power spectral density model by combining a periodic power spectral density and an aperiodic power spectral density, optimizing model parameters using a least square method to minimize a mean squared error, and obtaining a spectral curve fitted with power spectral densities; and
subtracting the aperiodic power spectral density from a fitted power spectrum to obtain the periodic power spectrum, and extracting the aperiodic component from an aperiodic fitting curve.

In an embodiment, calculating the phase-amplitude feature of the Beta-band signal of the LFP specifically includes the following steps:
acquiring an instantaneous phase of a low-frequency band of the Beta-band signal using Hilbert transform; and acquiring an instantaneous amplitude of a high-frequency band of the Beta-band signal using the Hilbert transform; and
establishing a relationship between a probability distribution of a high-frequency amplitude and a low-frequency phase using a preset method to obtain a PAC feature.

In an embodiment, the preset method for establishing the relationship between the probability distribution of the high-frequency amplitude and the low-frequency phase specifically includes the following steps:
dividing the low-frequency phase into n intervals of equal width;
calculating, within each phase interval, an average value of amplitudes of a high-frequency signal, constructing a distribution histogram of the amplitudes on phase, and normalizing the distribution histogram, an expression being as follows:

$${P}_{norm , i} = \frac{{P}_{i}}{{\sum }_{j} {P}_{j}}$$

where P_{norm,i} is a normalized probability distribution of an iₜₕ phase interval, Pᵢ is a frequency of an amplitude histogram within the iₜₕ phase interval, and Σⱼ Pⱼ is a sum of amplitude histograms within all phase intervals;
calculating a degree D_{KL} of deviation from a uniform distribution using a Kullback-Leibler (KL) distance, an expression being as follows:

$${D}_{KL} = {{\sum }_{i = 1}}^{N} {P}_{norm , i} log ( \frac{{P}_{norm , i}}{U} )$$

where N represents the number of intervals, and U represents the probability of the uniform distribution; and
calculating a PAC feature MI by normalizing the KL distance, an expression being as follows:

$$MI = \frac{{D}_{KL}}{log ( n )} .$$

In an embodiment, performing feature clustering analysis using the peak power spectral density, the periodic power spectrum, the aperiodic component, and the PAC feature specifically includes the following steps:
performing dimensionality reduction on the extracted peak power spectral density, the periodic power spectrum, the aperiodic component, and the PAC feature to obtain a dimension-reduced feature;
inputting the dimension-reduced feature into n clustering models, and outputting the sample typing results; and
calculating quality indicators of the sample typing results, evaluating clustering quality, and selecting an optimal clustering scheme.

In a second aspect of the disclosure, a PD sample feature typing system based on an LFP is provided, including a memory, and a processor. The memory includes a program for the PD sample feature typing method based on the LFP, and when the program for the PD sample feature typing method based on the LFP is executed by the processor, steps of the PD sample feature typing method based on the LFP are implemented.

In a third aspect of the disclosure provides a PD sample classification model training method based on an LFP is provided, which uses the typing results of the PD sample feature typing method based on the LFP for training, and includes the following steps:
using a sample rating scale as an input feature, training m classification models, classifying samples on the basis of the typing results, and searching for an optimal model configuration through hyperparameter optimization; and
validating and evaluating model performance using a leave-one-out cross-validation method, outputting a classification model having optimal performance, and validating interpretability of the classification model having optimal performance.

In an embodiment, a method for hyperparameter optimization search is Bayesian optimization.

In an embodiment, a method for validating the interpretability of the model includes the following steps:
using a SHapley Additive exPlanation (SHAP) method to calculate the impact of each input feature on model classification results, and outputting a SHAP importance ranking plot; and
validating the SHAP importance ranking plot by combining statistical analysis, and using inter-group difference significance to evaluate the impact of the feature on the classification results, to ensure the interpretability of the model.

Compared with the prior art, the technical solutions of the disclosure have the following advantageous effects.

The disclosure provides an integrated analysis method combining Beta-band features of the LFP of electroencephalogram with multidimensional clinical features, to achieve precise typing of PD samples. By extracting and integrating advanced neurophysiological features including the peak power spectral density, a periodic oscillating feature, an aperiodic background feature, and PAC, a multidimensional data model is constructed to enhance classification accuracy and reliability.

### BRIEF DESCRIPTION OF THE DRAWINGS

To make the objectives and technical solutions of the disclosure clearer, the disclosure provides the following drawings that are described as follows:

FIG. 1 is a flowchart of a typing method according to an embodiment of the disclosure;

FIG. 2 is a schematic diagram showing extraction of a peak power spectrum according to an embodiment of the disclosure;

FIG. 3 is a schematic diagram showing extraction of a periodic power spectrum and an aperiodic component according to an embodiment of the disclosure;

FIG. 4 is a comparison diagram showing indicators of different clustering methods according to an embodiment of the disclosure;

FIG. 5 is a comparison diagram showing clustering results according to an embodiment of the disclosure;

FIG. 6 is a comparison diagram showing prediction results of machine learning according to an embodiment of the disclosure; and

FIG. 7 is an analysis diagram showing the interpretability of a SHAP model according to an embodiment of the disclosure.

### DETAILED DESCRIPTION

For better understanding the above-described objectives, technical solutions and advantages of the disclosure, the disclosure is further described in detail below with reference to the accompanying drawings and specific implementations. It is to be noted that the embodiments of the present application and features therein can be combined with each other in the absence of conflict.

Many specific details are disclosed below to facilitate a thorough understanding of the disclosure. However, the disclosure can also be implemented in other ways different from those described herein. Therefore, the scope of protection of the disclosure is not limited to the specific embodiments disclosed below.

### Embodiment 1

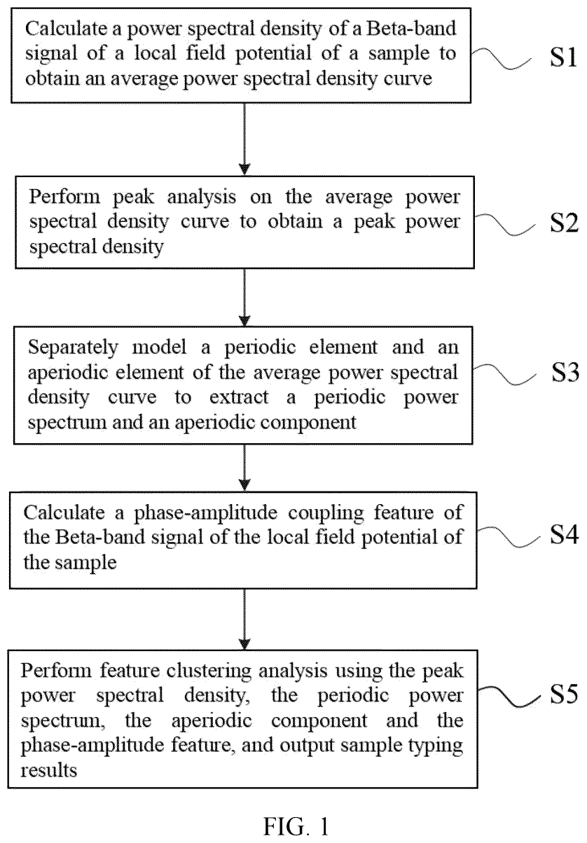

The disclosure provides a PD sample feature typing method based on an LFP, as shown in FIG. 1, which is a flowchart of the PD sample feature typing method based on the LFP, with the specific steps as follows.

S1: a power spectral density of a Beta-band signal of an LFP of a sample is calculated to obtain an average power spectral density curve.

More specifically, a method for calculating the power spectral density of the Beta-band signal of the LFP of the sample is a Welch method, with the specific process as follows.

An original LFP signal x[n] is framed, each frame being 1 second in length, and a windowed signal is calculated in combination with a Hanning window function w[n], an expression being as follows:

$${x}_{i} [ n ] = x [ n + i \cdot N ] \cdot w [ n ]$$

where n represents a sample index within a frame, n∈[1,N−1], xᵢ[n] represents a windowed signal of an iₜₕ frame, x[n] represents an original signal, w[n] represents a Hanning window, and N represents a frame length.

FFT is applied to each frame of the windowed signal to calculate a frequency-domain signal Xᵢ[f], an expression being as follows:

$${X}_{i} [ f ] = FFT ( {x}_{i} [ n ] )$$

where f represents a frequency, and xᵢ[n] represents a windowed signal of an iₜₕ frame.

A squared modulus of a frequency-domain signal for each frame is taken to obtain a power spectral density Pᵢ[f] of the iₜₕ frame, an expression being as follows:

$${P}_{i} [ f ] = {\vert {X}_{i} [ f ] \vert}^{2}$$

Power spectra of all frames are averaged to obtain an average power spectral density curve P_{avg}[f], an expression being as follows:

$${P}_{avg} [ f ] = \frac{1}{N} {{\sum }_{i = 1}}^{N} {P}_{i} [ f ]$$

where N is the number of frames.

S2: peak analysis is performed on the average power spectral density curve to obtain a peak power spectral density.

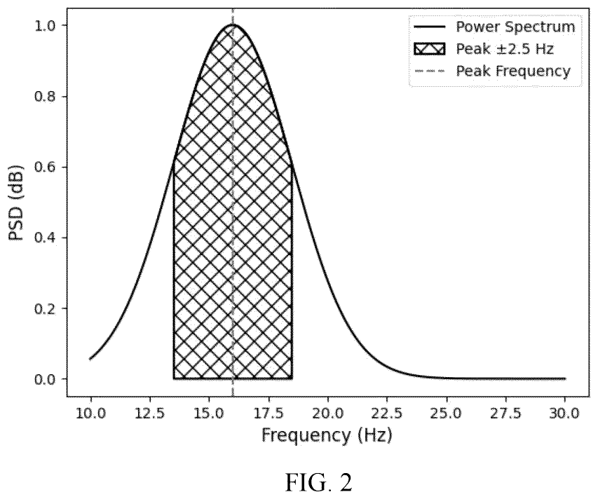

In this embodiment, a peak in a Beta band is first searched for. Subsequently, a band spanning 5 Hz on both left and right sides of the peak is calculated, and the peak power spectral density within this band is calculated. The reason for selecting this indicator is that it is a parameter consistently tracked over the long term in a sensing system and holds potential applications in adaptive deep brain stimulation (DBS), with a schematic diagram as shown in FIG. 2.

S3: a periodic element and an aperiodic element of the average power spectral density curve are separately modeled to extract a periodic power spectrum and an aperiodic component.

The specific process is as follows.

A periodic oscillating component of the power spectrum is fitted using a Gaussian function to obtain a periodic power spectral density P_{periodic}(f), an expression being as follows:

$${P}_{periodic} ( f ) = A \cdot {e}^{- \frac{{( f - CF )}^{2}}{2 \cdot {( BW / ( 2 \cdot \sqrt{2 \cdot ln ( 2 )} ) )}^{2}}}$$

where f represents a frequency, A represents an amplitude of an oscillation, CF represents a central frequency of the oscillation, and BW represents a bandwidth of the oscillation.

The aperiodic element of the power spectrum is fitted using a 1/f-like function to obtain an aperiodic power spectral density P_{aperiodic}(f), an expression being as follows:

$${P}_{aperiodic} ( f ) = \frac{offset}{{f}^{exponent}}$$

where f represents a frequency, offset represents an offset of the aperiodic element, and exponent represents an exponent of the aperiodic element.

A complete power spectral density model is constructed by combining a periodic power spectral density and an aperiodic power spectral density, model parameters are optimized using a least square method to minimize a mean squared error, and a spectral curve fitted with power spectral densities is obtained, an expression being as follows:

$$minimize {{\sum }_{i = 1}}^{N} {( {P}_{i} - P ( {f}_{i} ) )}^{2}$$

where Pᵢ is an actual power spectrum, P(f) is an initial fitting power spectrum of the model, and N is the number of frequency points.

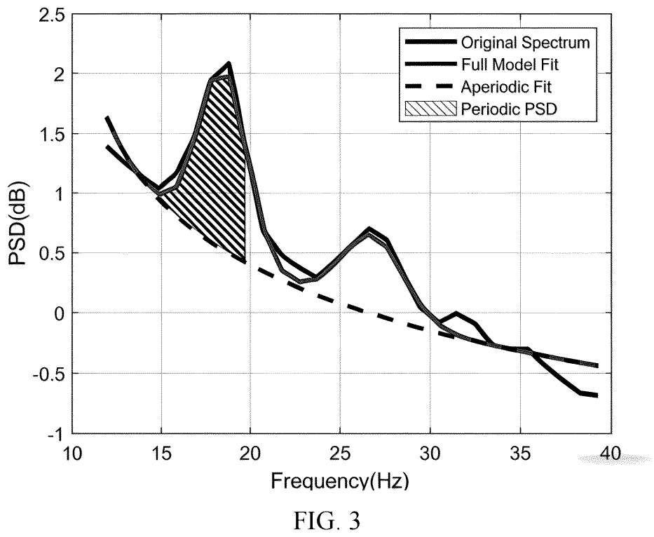

Subsequently, feature extraction is performed through power spectral parameterization. After parameterization of the power spectrum, a spectral curve fitted with power spectral densities can be obtained, and an aperiodic fitting curve can also be plotted, as shown in FIG. 3. By subtracting the aperiodic power spectral density (the area under the aperiodic fitting curve) from the fitted power spectrum, the periodic power spectrum (the shaded area in the figure) is obtained, and the aperiodic component (including offset and exponent) is extracted from the aperiodic fitting curve.

S3: a PAC feature of the Beta-band signal of the LFP of the sample is calculated.

The specific process is as follows.

An instantaneous phase φ(t) of a low-frequency band of the Beta-band signal is acquired using Hilbert transform, an expression being as follows:

$$ϕ ( t ) = arg [ Hilbert ( low - frequency Beta signal ) ]$$

An instantaneous amplitude |Aₕ(t)| of a high-frequency band (300-400 Hz, and 400-500 Hz) of the Beta-band signal is acquired using the Hilbert transform, an expression being as follows:

$$\vert {A}_{h} ( t ) \vert = \vert Hilbert ( high - frequency ) HFO signal ) \vert$$

A relationship between a probability distribution of a high-frequency amplitude and a low-frequency phase is established using a preset method to obtain the PAC feature, with the following steps specifically included.
n intervals of equal width are delineated in a low-frequency phase, and 18 intervals are delineated in this embodiment, respectively corresponding to each 200 within [0, 2π]. Within each phase interval, an average value of high-frequency signal amplitudes is calculated, a distribution histogram of the amplitudes on phase is constructed, and the distribution histogram is normalized, an expression being as follows:

$${P}_{n o rm , i} = \frac{{P}_{i}}{{\sum }_{j} {P}_{j}}$$

where P_{norm,i} is a normalized probability distribution of an iₜₕ phase interval, Pᵢ is a frequency of an amplitude histogram within the iₜₕ phase interval, and Σⱼ Pⱼ is a sum of amplitude histograms within all phase intervals.

A degree D_{KL} of deviation from a uniform distribution is calculated using a KL distance, an expression being as follows:

$${D}_{KL} = {\sum }_{j = 1}^{N} {P}_{n o rm , i} log ( \frac{{P}_{n o rm , i}}{U} )$$

where N represents the number of intervals, and U represents the probability of the uniform distribution, typically being 1/N.

A PAC feature MI is calculated by normalizing the KL distance, an expression being as follows:

$$MI = \frac{{D}_{K L}}{log ( n )}$$

S4: feature clustering analysis is performed using the peak power spectral density, the periodic power spectrum, the aperiodic component, and the PAC feature, and sample typing results are outputted.

In this embodiment, the specific process is as follows.

Principal component analysis (PCA) is applied to perform dimensionality reduction on the extracted peak power spectral density, periodic power spectrum, aperiodic component, and PAC feature of samples from 55 patients to obtain a dimension-reduced feature.

The dimension-reduced feature is inputted into n clustering models separately. In this embodiment, four clustering methods are considered, including hierarchical clustering (HAC), K-Means, Bisecting K-Means (BiKMeans) and hierarchical density-based spatial clustering of applications with noise (HDBSCAN). The number of clusters k is 2, and sample typing results are outputted.

A silhouette coefficient and a Davies-Bouldin (DB) index of the sample typing results are calculated, clustering quality is evaluated, and an optimal clustering scheme is selected.

For silhouette coefficient, the quality of a cluster is measured by calculating the degree of similarity between a point and its own cluster, and then this result is averaged across the entire dataset. A score close to −1 indicates a meaningless group, while a score close to +1 represents a well-separated and compact group, with an expression as follows:

$$Silhouette = \frac{1}{N} {\sum }_{i = 1}^{N} \frac{b ( i ) - a ( i )}{max \{ a ( i ) , b ( i ) \}}$$

where a(i) is an average distance between an iₜₕ data point and all other points within the same cluster, b(i) represents a smallest average distance between the iₜₕ data point and other points in different clusters, and N is the total number of data points.

DB index is also an index for evaluating clustering performance, reflecting the balance between intra-cluster compactness and inter-cluster separation, with an expression as follows:

$$D B = \frac{1}{k} {\sum }_{i = 1}^{k} {max}_{j \neq i} ( \frac{{σ}_{i} + {σ}_{j}}{{d}_{ij}} )$$

where k is the number of clusters, σᵢ and σⱼ are average intra-cluster distances for a cluster i and a cluster j, respectively, representing an average distance from samples in a cluster to a centroid; and dg is a Euclidean distance between centroids of the cluster i and the cluster j. The compactness and separation between clusters can be evaluated by calculating a maximum similarity index between each cluster and all other clusters, and then taking the average of these maximum values. A smaller DB index indicates that intra-cluster samples are closer and inter-cluster separation is larger, and the clustering performance is better. A higher DB index indicates poorer clustering performance.

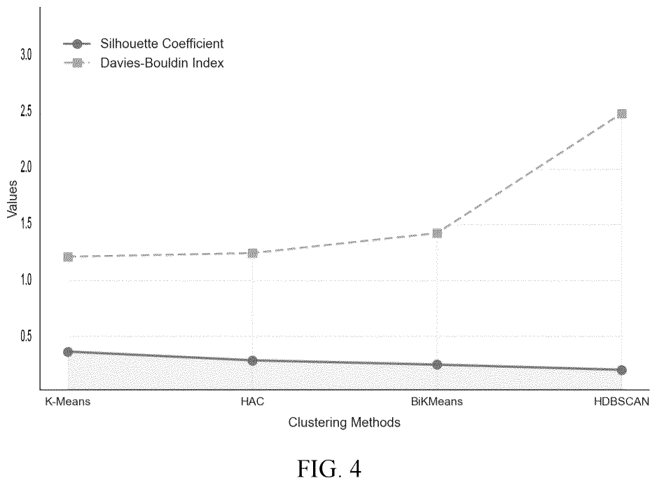

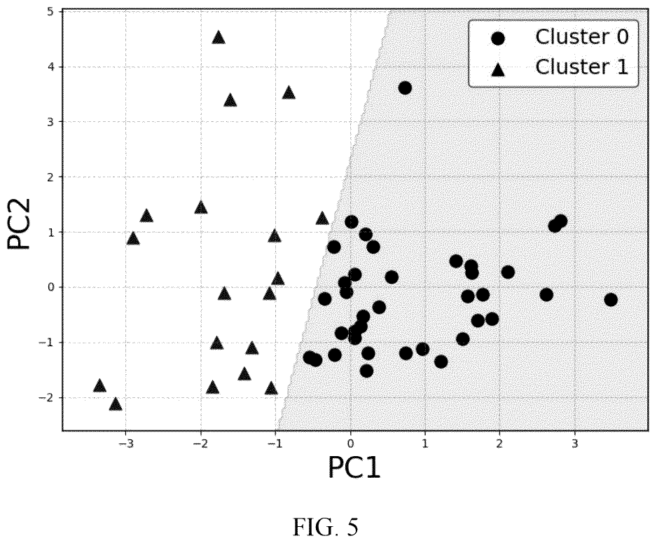

By comparing the clustering results of different methods, an optimal clustering method is selected. The clustering index results of the disclosure are as shown in FIG. 4. In summary, K-Means has the optimal performance. The final clustering results are as shown in FIG. 5, with one cluster defined as Cluster 0 (CL0) and the other as Cluster 1 (CL1).

### Embodiment 2

This embodiment provides a PD sample feature typing system based on an LFP, including a memory, and a processor. The memory includes a program for a PD sample feature typing method based on an LFP, and when the program for the PD sample feature typing method based on the LFP is executed by the processor, steps of the PD sample feature typing method based on the LFP as described in Embodiment 1 are implemented.

### Embodiment 3

This embodiment provides a PD sample classification model training method based on an LFP, which uses the typing results of the PD sample feature typing method based on the LFP as described in Embodiment 1 for training, and includes the following steps.

Using Part I, Part II, Part III, and Part IV of the movement disorder society-unified PD rating scale (MDS-UPDRS), and mini-mental state examination (MMSE) and a 24-item Hamilton depression rating scale (HAMD-24) as input features, different classification models are trained separately, including Logistic Regression, adaptive boosting (AdaBoost), support vector machine (SVM), Naive Bayes, Extreme Gradient Boosting (XGBoost), categorical boosting (CatBoost), K-nearest neighbors (KNN), and Multi-Layer Perceptron (MLP). The samples are classified according to the newly identified clusters CL1 and CL0 mentioned in the clustering analysis. To ensure that each model achieves optimal performance, Bayesian optimization is employed to search for optimal hyperparameters for each classifier, thereby fully leveraging the predictive capabilities of the models.

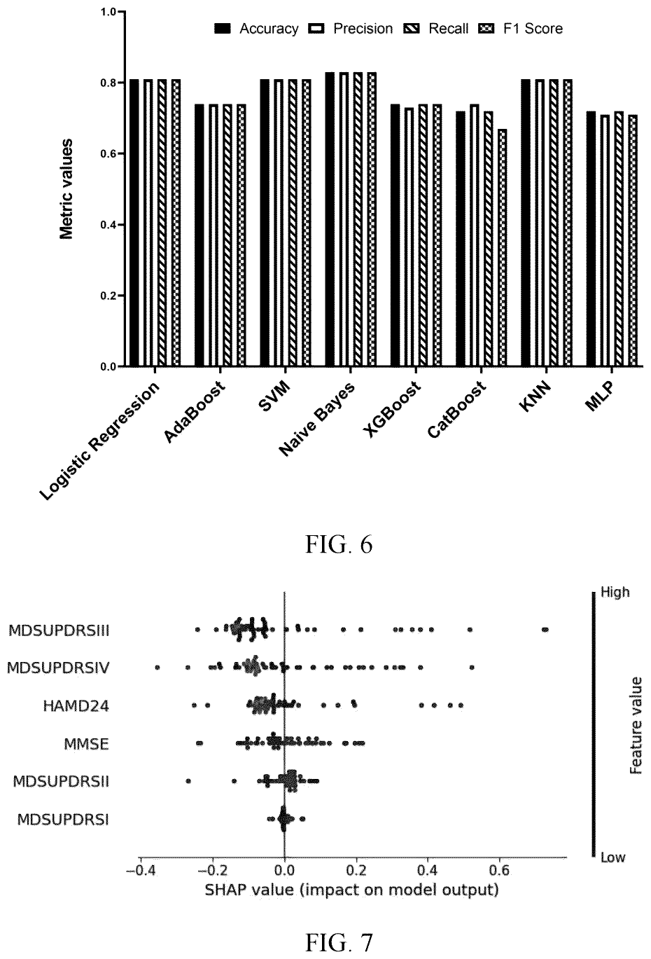

Model performance is validated and evaluated using a leave-one-out cross-validation method, an accuracy rate, a recall rate and an F1 score are calculated, a classification model having optimal performance is outputted, and interpretability of the classification model having optimal performance is validated. This validation method is particularly effective when processing small-scale medical datasets, as it ensures that the model is tested on completely unseen sample data, thereby providing a more realistic and reliable assessment of the generation capabilities of the models. The evaluation results of a plurality of models are as shown in FIG. 6.

A model, Naive Bayes, which has the optimal overall performance, is selected, achieving an accuracy rate, precision degree, recall score, and F1 score of 87%.

More specifically, a method for validating the interpretability of the model includes the following steps.

A SHAP method is used to calculate the impact of each input feature on the classification results of the model. The input feature includes MDS-UPDRS, MMSE, HAMD-24, MDS-UPDRSI, MDS-UPDRSII, MDS-UPDRSIII, MDS-UPDRSIV, and MoCA. A SHAP importance ranking plot is outputted to quantify the contribution of different features.

The SHAP importance ranking plot is validated by combining statistical analysis. The SHAP analysis results are as shown in FIG. 7, which clearly shows the contribution of each feature in the model. Finally, statistical analysis is performed on all clinical features of the two clusters to further explore the relationship between the clustering results and clinical variables. By inter-group difference tests for each feature, it is found that multiple clinical features in the two sample groups have significant differences. These differences are highly consistent with the feature importance ranking derived from the previous SHAP analysis. Table 1 shows the inter-group differences in clinical scores across the new typing.

TABLE 1

| Clinical Features | CL0 | CL1 | P-value |
|---|---|---|---|
| MDS-UPDRSIII | 38.703 ± 11.813 | 54.167 ± 18.573 | <0.001 *** |
| MDS-UPDRSIV | 3.649 ± 3.482 | 8.222 ± 3.813 | <0.001 *** |
| HAMD-24 | 8.054 ± 4.66 | 12.167 ± 6.373 | 0.009 ** |
| MMSE | 25.322 ± 2.224 | 26.782 ± 1.957 | 0022* |
| MDS-UPDRSII | 18.622 ± 8.281 | 20.444 ± 6.07 | 0.410 |
| MDS-UPDRSI | 12.676 ± 5.647 | 13.056 ± 6.159 | 0.821 |

Specifically, features that show high importance in SHAP analysis, such as MIDS-UPDRSIII and MIDS-UPDRSIV, also show highly significant differences between groups in statistical analysis. This indicates mutual validation between the key variables focused by the model during prediction and the actual clinical features. Moreover, the inter-group difference of HAM/A and MMSE also shows statistical significance, which further supports the contribution of these variables to the classification prediction of the model.

Obviously, the above-described embodiments of the disclosure are merely used to clearly describe the disclosure, rather than limiting the implementation methods of the disclosure. For those ordinary skilled in the art, various other forms of changes or modifications can be made on the basis of the above description. It is neither necessary nor feasible to exhaustively list all possible implementation methods herein. Any modifications, equivalents and improvements made within the spirit and principle of the disclosure are included in the scope of protection of the claims of the disclosure.

1. A Parkinson's disease sample feature typing method based on a local field potential, comprising the following steps:
calculating a power spectral density of a Beta-band signal of a local field potential of a sample to obtain an average power spectral density curve;
performing peak analysis on the average power spectral density curve to obtain a peak power spectral density;
separately modeling a periodic element and an aperiodic element of the average power spectral density curve to extract a periodic power spectrum and an aperiodic component;
acquiring an instantaneous phase of a low-frequency band of the Beta-band signal of the local field potential of the sample using Hilbert transform; and acquiring an instantaneous amplitude of a high-frequency band of the Beta-band signal using the Hilbert transform;
establishing a relationship between a probability distribution of a high-frequency amplitude and a low-frequency phase using a preset method to obtain a phase-amplitude coupling feature;
performing dimensionality reduction on the extracted peak power spectral density, the periodic power spectrum, the aperiodic component, and the phase-amplitude coupling feature to obtain a dimension-reduced feature;
inputting the dimension-reduced feature into n clustering models, and outputting sample typing results; and
calculating quality indicators, comprising a silhouette coefficient and a Davies-Bouldin index, for the sample typing results, evaluating clustering quality using the quality indicators, selecting an optimal clustering scheme, and outputting final sample typing results.

2. The Parkinson's disease sample feature typing method based on the local field potential according to claim 1, wherein a method for calculating the power spectral density of the Beta-band signal of the local field potential of the sample is a Welch method, comprising the following steps:
framing an original local field potential signal x[n], and calculating a windowed signal in combination with a Hanning window function w[n], an expression being as follows:
$${x}_{i} [ n ] = x [ n + i \cdot N ] \cdot w [ n ]$$
where n represents a sample index within a frame, n∈[1,N−1], xᵢ[n] represents a windowed signal of an iₜₕ frame, x[n] represents an original signal, w[n] represents a Hanning window, and N represents a frame length;
applying discrete Fourier transform to each frame of the windowed signal, and calculating a frequency-domain signal Xᵢ[f], an expression being as follows:
$${X}_{i} [ f ] = FFT ( {x}_{i} [ n ] )$$
where f represents a frequency, FFT represents discrete Fourier transform, and xᵢ[n] represents a windowed signal of an iₜₕ frame;
taking a squared modulus of a frequency-domain signal for each frame to obtain a power spectral density Pᵢ[f] of the iₜₕ frame, an expression being as follows:
$${P}_{i} [ f ] = {\vert {X}_{i} [ f ] \vert}^{2} ;$$
and
averaging power spectra of all frames to obtain an average power spectral density curve P_{avg}[f], an expression being as follows:
$${P}_{avg} [ f ] = \frac{1}{N} {\sum }_{j = 1}^{N} {P}_{i} [ f ]$$
where N is the number of frames.

3. The Parkinson's disease sample feature typing method based on the local field potential according to claim 1, wherein separately modeling the periodic element and the aperiodic element of the average power spectral density curve to extract the periodic power spectrum and the aperiodic component comprises the following steps:
fitting a periodic oscillating component of the power spectrum using a Gaussian function to obtain a periodic power spectral density P_{periodic}(f), an expression being as follows:
$${P}_{periodic} ( f ) = A \cdot {e}^{- \frac{{( f - CF )}^{2}}{2 \cdot {( BW / ( 2 \sqrt{2 \cdot ln ( 2 )} ) )}^{2}}}$$
where f represents a frequency, A represents an amplitude of an oscillation, CF represents a central frequency of the oscillation, and BW represents a bandwidth of the oscillation;
fitting the aperiodic element of the power spectrum using a 1/f-like function to obtain an aperiodic power spectral density P_{aperiodic}(f), an expression being as follows:
$${P}_{aperiodic} ( f ) = \frac{offset}{{f}^{exponent}}$$
where f represents a frequency, offset represents an offset of the aperiodic element, and exponent represents an exponent of the aperiodic element;
constructing a complete power spectral density model by combining a periodic power spectral density and an aperiodic power spectral density, optimizing model parameters using a least square method to minimize a mean squared error, and obtaining a spectral curve fitted with power spectral densities; and
subtracting the aperiodic power spectral density from a fitted power spectrum to obtain the periodic power spectrum, and extracting the aperiodic component from an aperiodic fitting curve.

4. The Parkinson's disease sample feature typing method based on the local field potential according to claim 1, wherein the preset method for establishing the relationship between the probability distribution of the high-frequency amplitude and the low-frequency phase comprises the following steps:
dividing the low-frequency phase into n intervals of equal width;
calculating, within each phase interval, an average value of amplitudes of a high-frequency signal, constructing a distribution histogram of the amplitudes on phase, and normalizing the distribution histogram, an expression being as follows:
$${P}_{norm , j} = \frac{{P}_{i}}{{\sum }_{j} {P}_{j}}$$
where P_{norm,i} is a normalized probability distribution of an iₜₕ phase interval, Pᵢ is a frequency of an amplitude histogram within the iₜₕ phase interval, and Σⱼ Pⱼ is a sum of amplitude histograms within all phase intervals;
calculating a degree D_{KL} of deviation from a uniform distribution using a Kullback-Leibler (KL) distance, an expression being as follows:
$${D}_{KL} = {\sum }_{i = 1}^{N} {P}_{norm , i} log ( \frac{{P}_{norm , i}}{U} )$$
where N represents the number of intervals, and U represents the probability of the uniform distribution; and
calculating a phase-amplitude coupling feature MI by normalizing the KL distance, an expression being as follows:
$$MI = \frac{{D}_{KL}}{log ( n )} .$$

5. A Parkinson's disease sample feature typing system based on a local field potential, comprising a memory, and a processor, wherein the memory comprises a program for the Parkinson's disease sample feature typing method based on the local field potential, and when the program for the Parkinson's disease sample feature typing method based on the local field potential is executed by the processor, steps of the Parkinson's disease sample feature typing method based on the local field potential according to claim 1 are implemented.

6. A Parkinson's disease sample feature typing system based on a local field potential, comprising a memory, and a processor, wherein the memory comprises a program for the Parkinson's disease sample feature typing method based on the local field potential, and when the program for the Parkinson's disease sample feature typing method based on the local field potential is executed by the processor, steps of the Parkinson's disease sample feature typing method based on the local field potential according to claim 2 are implemented.

7. A Parkinson's disease sample feature typing system based on a local field potential, comprising a memory, and a processor, wherein the memory comprises a program for the Parkinson's disease sample feature typing method based on the local field potential, and when the program for the Parkinson's disease sample feature typing method based on the local field potential is executed by the processor, steps of the Parkinson's disease sample feature typing method based on the local field potential according to claim 3 are implemented.

8. A Parkinson's disease sample feature typing system based on a local field potential, comprising a memory, and a processor, wherein the memory comprises a program for the Parkinson's disease sample feature typing method based on the local field potential, and when the program for the Parkinson's disease sample feature typing method based on the local field potential is executed by the processor, steps of the Parkinson's disease sample feature typing method based on the local field potential according to claim 4 are implemented.

9. A Parkinson's disease sample classification model training method based on a local field potential, using the typing results of the Parkinson's disease sample feature typing method based on the local field potential according to claim 1 for training, and comprising the following steps:
using a sample rating scale as an input feature, training m classification models, classifying samples on the basis of the typing results, and searching for an optimal hyperparameter; and
validating and evaluating model performance using a leave-one-out cross-validation method, outputting a classification model having optimal performance, and validating interpretability of the classification model having optimal performance.

10. A Parkinson's disease sample classification model training method based on a local field potential, using the typing results of the Parkinson's disease sample feature typing method based on the local field potential according to claim 2 for training, and comprising the following steps:
using a sample rating scale as an input feature, training m classification models, classifying samples on the basis of the typing results, and searching for an optimal hyperparameter; and
validating and evaluating model performance using a leave-one-out cross-validation method, outputting a classification model having optimal performance, and validating interpretability of the classification model having optimal performance.

11. A Parkinson's disease sample classification model training method based on a local field potential, using the typing results of the Parkinson's disease sample feature typing method based on the local field potential according to claim 3 for training, and comprising the following steps:
using a sample rating scale as an input feature, training m classification models, classifying samples on the basis of the typing results, and searching for an optimal hyperparameter; and
validating and evaluating model performance using a leave-one-out cross-validation method, outputting a classification model having optimal performance, and validating interpretability of the classification model having optimal performance.

12. A Parkinson's disease sample classification model training method based on a local field potential, using the typing results of the Parkinson's disease sample feature typing method based on the local field potential according to claim 4 for training, and comprising the following steps:
using a sample rating scale as an input feature, training m classification models, classifying samples on the basis of the typing results, and searching for an optimal hyperparameter; and
validating and evaluating model performance using a leave-one-out cross-validation method, outputting a classification model having optimal performance, and validating interpretability of the classification model having optimal performance.

13. The Parkinson's disease sample classification model training method based on the local field potential according to claim 9, wherein a method for validating the interpretability of the model comprises the following steps:
using a SHapley Additive exPlanation (SHAP) method to calculate the impact of each input feature on model classification results, and outputting a SHAP importance ranking plot; and
validating the SHAP importance ranking plot by combining statistical analysis, and using inter-group difference significance to evaluate the impact of the feature on the classification results, to ensure the interpretability of the model.
